(19) United States
(12) Patent Application Publication
(10) Pub. No.: US 2004/0166130 A1
(43) Pub. Date: Aug. 26, 2004

(54) Lanolin-free cosmetic composition comprising a hydroxylated fatty acid aromatic ester

(75) Inventors: Vanina Filippi, Paris (FR); Sophie Salem, Villejuif (FR); Frederic Auguste, Chevilly-Larue (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/743,084

(22) Filed: Dec. 23, 2003

Related U.S. Application Data
(60) Provisional application No. 60/438,772, filed on Jan. 9, 2003.

(30) Foreign Application Priority Data
Dec. 23, 2002 (FR) 0216533

(51) Int. Cl. A61K 7/06

(52) U.S. Cl. 424/401

(57) ABSTRACT

The invention relates to a care or make-up cosmetic composition, devoid of lanolin or of lanolin derivatives, comprising at least one ester which is liquid at ambient temperature resulting from the esterification by an aromatic acid of at least one pendent hydroxyl group or hydroxyl group at the chain end of a hydroxylated aliphatic compound.

### REFERENCE TO PRIOR APPLICATIONS

This application claims priority to U.S. provisional application 60/438,772 filed Jan. 9, 2003, and to French patent application 0216533 filed Dec. 23, 2002, both incorporated herein by reference.

### FIELD OF THE INVENTION

The present invention relates to a cosmetic composition for making up or caring for the skin, including the scalp, both of the human face and of the human body, the lips or the superficial body growths of human beings, such as the hair, eyelashes, eyebrows or nails, comprising a cosmetically acceptable medium comprising an aliphatic ester of specific ester. This composition is devoid of lanolin and it comprises one or more esters resulting from the complete or partial esterification of a hydroxylated aliphatic compound with an aromatic acid. This composition has noteworthy cosmetic properties and confers in particular on the make-up or care composition properties of gloss, of slip on application, of comfort and of non-stickiness.

The composition of the invention can in particular constitute a product for making up the body, lips or superficial body growths of human beings having in particular non-therapeutic care and/or treatment properties. It constitutes in particular a lipstick, a lip gloss, a face powder, an eyeshadow, a tattooing product, a mascara, an eyeliner, a nail varnish, a product for artificial tanning of the skin, or a product for colouring or caring for the hair.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

There exist numerous cosmetic compositions for which the properties of gloss of the film deposited, after application to keratinous substances (skin, lips, superficial body growths), are desirable. Mention may be made, for example, of lipsticks, nail varnishes or certain hair products. From this perspective, the formulator has available several types of starting materials and in particular lanolins, used in combination with “glossy” oils, such as oily polymers, for example a) polybutenes, which have a high viscosity (typically of greater than 2 819 Pa·s at 23° C., measured with a Brookfield RV viscometer equipped with a No. 1 needle rotating at 0.5 rev/min), b) fatty alcohol or acid esters, the carbon number of which is high (typically greater than 16), or c) certain vegetable oils.

The esters resulting from the partial or complete esterification of a hydroxylated aliphatic compound with an aromatic acid, as disclosed in Patent Application EP 1 097 699, confer, on cosmetic products, good properties of application, of gloss and of dispersion of the pigments. The formulations disclosed in this document all comprise lanolin for contributing creaminess and comfort to these compositions. However, the lanolins exhibit the disadvantage of being sensitive to heat and to ultraviolet radiation and have a tendency to oxidize over time with release of an unpleasant smell, which limits their use in cosmetic compositions. Furthermore, when lanolins are used in combination with oils commonly used in the cosmetics field, the compositions obtained exhibit problems of stickiness, which become more pronounced as the oil used increases in viscosity.

### DETAILED DESCRIPTION OF THE PREFERRED EMBODIMENTS

The invention has specifically as subject-matter a cosmetic composition for caring for or making up keratinous substances and in particular the skin and/or lips and/or superficial body growths which makes it possible to overcome the above-mentioned disadvantages.

Surprisingly, the inventors have found that it is possible to obtain a composition comprising one or more esters resulting from the complete or partial esterification of a hydroxylated aliphatic compound with an aromatic acid which is glossy, comfortable and non-sticky and which does not comprise lanolin.

In addition, this composition exhibits good dispersion of the pigments and/or of the fillers present in the composition, it does not exude when it is in the form of a stick, it exhibits good properties of spreading and of slip and additionally confers, on the film deposited, properties of gloss, of comfort, of good hold over time (no change in colour for at least 3 hours, homogeneous disappearance of the make-up), of non-stickiness and of non-greasiness. In addition, it is stable, in particular for several months at ambient temperature (25° C. for more than a year) but also towards heat (47° C. for 2 months) and towards ultraviolet radiation, without deterioration in the smell over time.

More specifically, a subject-matter of the invention is a cosmetic composition for caring for or making up keratinous substances, comprising at least one ester possessing an aromatic group which is liquid at ambient temperature resulting from the esterification by an aromatic acid of at least one pendent hydroxyl group or hydroxyl group at the chain end of a hydroxylated aliphatic compound chosen from hydroxylated aliphatic acids and their esters, the said composition being devoid of lanolin or of lanolin derivatives.

The term “lanolin derivatives” is understood to mean in particular oxypropylenated lanolin, acetylated lanolin or isopropyl lanolate, for example the oxypropylenated (5 PO) lanolin wax sold under the reference Emery 1695 by Cognis.

The composition according to the invention advantageously comprises at least one pasty compound other than lanolin or than its derivatives.

Ester Possessing an Aromatic Group

The composition comprises at least one ester possessing an aromatic group which is liquid at ambient temperature resulting from the esterification by an aromatic acid of at least one pendent hydroxyl group or hydroxyl group at the chain end of a hydroxylated aliphatic compound chosen from hydroxylated aliphatic acids or their esters.

The term “hydroxylated aliphatic compound” is understood to mean an aliphatic hydroxycarboxylic acid or an aliphatic hydroxycarboxylic acid ester. The acid (non-esterified) comprises in particular from 2 to 40 carbon atoms, preferably from 10 to 34 carbon atoms and better still from 12 to 28 carbon atoms; in addition, it comprises from 1 to 20 hydroxyl groups, preferably from 1 to 10 hydroxyl groups and better still from 1 to 6 hydroxyl groups capable of being esterified by the aromatic acid. The hydroxylated compound in the ester form results from the esterification of the —COOH functional group of an aliphatic hydroxycarboxylic acid by an aliphatic alcohol which can comprise from 1 to 40 carbon atoms and better still from 3 to 30 carbon atoms. This alcohol can be a monoalcohol or a polyol. The esters resulting from the reaction of the aliphatic hydroxycarboxylic acid with a polyol can be partially or completely esterified esters.

Preferably, the hydroxylated aliphatic compound is chosen from the esters resulting from an aliphatic hydroxycarboxylic acid. In other words, the liquid aromatic ester of the composition of the invention is an ester ester. Advantageously, this aromatic ester is a fatty acid ester ester, the fatty acid residue of which comprises at least 12 carbon atoms. Preferably, the hydroxyl group involved in the esterification by the aromatic acid is carried on the acid part of the hydroxylated compound.

According to the invention, the composition can comprise one or more aromatic esters which are liquid at ambient temperature (25° C.) and atmospheric pressure (760 mmHg).

When the hydroxyl group of the hydroxylated aliphatic compound involved in the esterification with the aromatic acid is at the chain end, this group is in the α,ω position with respect to the —COOH functional group of the aliphatic hydroxycarboxylic acid.

Preferably, the aromatic esters according to the invention exhibit a viscosity of greater than 500 cP (50 Pa·s) at 20° C. but also at 23° C., preferably ranging from 900 to 10 000 cP (90 to 1 000 Pa·s) and better still from 950 to 5 000 cP (95 to 500 Pa·s), measured in particular with a Brookfield RV type viscometer or a Brookfield “DV-II+” type viscometer of LV type equipped with a No. 1 needle rotating at between 0.5 and 10 rev/min, after 10 minutes and/or a refractive index ≧1.48 at 20° C. and in particular ranging from 1.48 to 1.55 (the refractive index being defined for the D line of sodium).

The aromatic acid can be chosen from the following carboxylic acids:

a) monoacids, such as benzoic acid, phenylacetic acid, cinnamic acid, 3-phenylpropanoic acid or salicylic acid;

b) diacids, such as terephthalic acid;

c) triacids, such as trimellitic acid; and

d) tetraacids, such as pyromellitic acid.

Advantageously, the aromatic carboxylic acid is benzoic acid.

Mention may be made, as ester which can be used in the invention, of those resulting from the esterification by at least one aromatic acid of at least one of the following hydroxylated aliphatic carboxylic acids:

i) saturated linear monohydroxylated monoacids of formula:

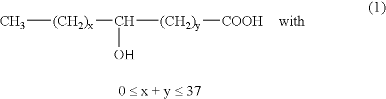

such as lactic acid (x+y=0); 12-hydroxyoctadecanoic (or 12-hydroxystearic) acid of formula:

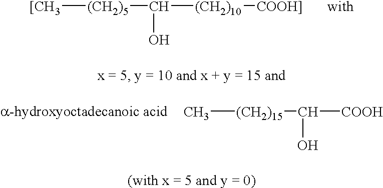

or (2) HO—CH₂—(CH₂)ₙ—COOH with 0≦x≦38

such as glycolic acid HO—CH₂—COOH with x=0; or juniperic acid (16-hydroxyhexadecanoic acid) of formula HO—CH₂—(CH₂)₁₄—COOH with x=14;

ii) saturated branched monohydroxylated monoacids of formula:

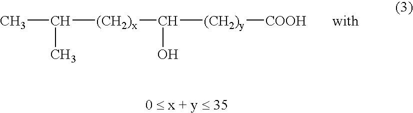

such as 5-methyl-2-hydroxyhexanoic acid (leucinic acid) of formula:

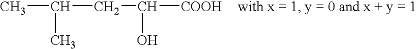

or 2-ethyl-3-hydroxycaprylic acid of formula:

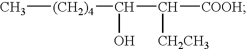

iii) unsaturated monohydroxylated monoacids of formula:

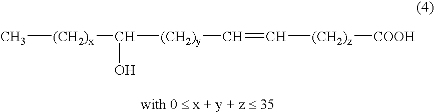

such as cis-12-hydroxy-9-octadecanoic acid (or ricinoleic acid) of formula:

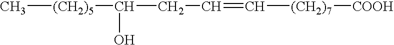

with x=5, y=1, z=7 and x+y+z=13

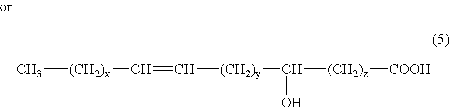

with 0≦x+y+z≦35

such as 3-hydroxy-4-hexenoic acid of formula

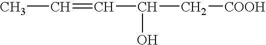

with x=0, y=0, z=1 and x+y+z=1

or 2-hydroxy-15-tetracosenoic acid (or hydroxynervonic acid) of formula:

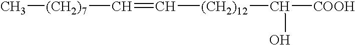

with x=7, y=12, z=0 and x+y+z=17

or (6) HOCH₂—(CH₂)ₓ—CH═CH—(CH₂)_{y}—COOH with 0≦x+y≦36 such as 16-hydroxy-6-hexadecenoic acid with x=8, y=4 and x+y=12

of formula HO—CH₂—(CH₂)₈—CH═CH—(CH₂)₄—COOH;

iv) polyhydroxylated monoacids of formula:

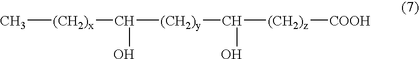

with 0≦x+y+z≦36

such as 9,10-dihydroxyoctadecanoic acid of formula

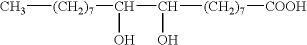

with x=7, y=0, z=7 and x+y+z=14

such as 9,12-dihydroxyoctadecanoic acid of formula (7) with x=5, y=2, z=7 and x+y+z=14

or 9,10,16-trihydroxyhexadecanoic acid (aleuritic acid), 9,10,12-trihydroxyoctadecanoic acid of formula

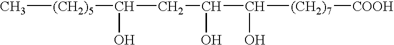

or hexahydroxyoctadecanoic acid and octahydroxyoctadecanoic acid;

v) saturated monohydroxylated polyacids of formula:

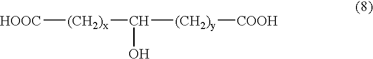

with 0≦x+y≦37, such as malic acid,

or citric acid; and

vi) saturated polyhydroxylated polyacids, such as tartaric acid;

and their mixtures.

Mention may be made, as other hydroxylated aliphatic compound which can be used in the invention, of those resulting from the esterification by at least one aromatic acid of at least one of the following esters of hydroxylated aliphatic acids:

vii) esters of saturated linear monohydroxylated aliphatic monoacids, such as:

esters of lactic acid, such as isostearyl lactate, the lactate resulting from C₁₂-C₁₃ alcohol, octyldodecyl lactate, oleyl lactate or myristyl lactate;

esters of 12-hydroxyoctadecanoic (or 12-hydroxystearic) acid, such as 2-ethylhexyl hydroxystearate, octyldodecyl hydroxystearate, isostearyl hydroxystearate, isodecyl hydroxystearate, glyceryl trihydroxystearate (or hydrogenated castor oil) or dipentaerythrityl hexahydroxystearate;

viii) esters of unsaturated monohydroxylated aliphatic monoacids, such as esters of ricinoleic acid (or cis-12-hydroxy-9-octadecenoic acid), such as butyl ricinoleate, octyldodecyl ricinoleate, cetyl ricinoleate or glyceryl triricinoleate (castor oil);

ix) esters of saturated monohydroxylated aliphatic polyacids, such as diisostearyl malate, triisostearyl citrate or trioctyldodecyl citrate;

x) esters of saturated polyhydroxylated aliphatic polyacids, such as the tartrate resulting from the reaction with 2 branched C₁₂-C₁₋₃ alcohols;

and their mixtures.

Mention may generally be made, as hydroxylated compound in the ester form which can be used in the invention and which results from the esterification of a polyol, of:

xi) partial or complete esters of C₂ to C₁₆ polyol which has reacted with a hydroxylated aliphatic acid, such as, in particular, triglycerides, esters of pentaerythritol, of neopentyl glycol, of dipentaerythritol or of polyglycerol, or sorbitol esters;

and their mixtures.

Preferably, the aromatic esters of the invention are chosen from aliphatic fatty acid ester esters, the fatty acid residue of which comprises at least 12 carbon atoms. In particular, the hydroxylated compound is chosen from esters of ricinoleic acid, esters of 12-hydroxystearic acid, esters of lactic acid, esters of 14-hydroxyicosenoic acid, and their mixtures.

Use is made in particular, as aromatic ester, of:

the ester resulting from the esterification reaction of castor oil with benzoic acid in the proportions of 1 to 1 (1/1), for example sold by Finetex under the reference Finsolv BCO-110, and which will be referred to subsequently as glyceryl monobenzoyl ricinoleate,

the compound resulting from the reaction of castor oil with benzoic acid in the proportions of 1 to 1.5 (1/1.5), for example sold by Finetex under the reference Finsolv BCO-115, and which will also be referred to subsequently as glyceryl mono/dibenzoyl ricinoleate,

the compound resulting from the esterification reaction of castor oil with benzoic acid in the proportions of 1 to 2 (½), for example sold by Finetex under the reference Finsolv BCO-120, and which will be referred to subsequently as glyceryl dibenzoyl ricinoleate,

the compound resulting from the esterification reaction of castor oil with benzoic acid in the proportions of 1 to 3 (⅓), for example sold by Finetex under the reference Finsolv BCO-130, and which will be referred to subsequently as glyceryl tribenzoyl ricinoleate, and

their mixtures.

Ricinoleic acid represents from 80 to 92% of castor oil. Thus, its esterification results predominantly (80 to 92%) in the ester of the ester of ricinoleic acid.

Use may also be made of the ester resulting from the esterification reaction with benzoic acid of the oil predominantly comprising (52 to 57%) an ester of 14-hydroxyicosenoic acid or lesquerolic acid.

The aromatic ester of the composition of the invention can be manufactured according to the process disclosed in the document U.S. Pat. No. 5,959,130.

The aromatic ester of the composition of the invention can represent from 0.1 to 99.9% of the total weight of the composition, preferably from 1 to 99%, better still from 1 to 80%, even better still from 10 to 40% and even better still from 15 to 25%, and can generally be present in an amount sufficient to confer, on the composition, properties of non-greasiness, of non-stickiness, of slip, of gloss, of coverage, of stability and/or of hold over time.

Pasty

The term “pasty”, within the meaning of the present invention, is understood to mean a lipophilic fatty compound with a reversible solid/liquid change of state exhibiting, in the solid state, an anisotropic crystalline arrangement and comprising, at a temperature of 23° C., a liquid fraction and a solid fraction.

The term “pasty compound”, within the meaning of the invention, is understood to mean a compound having a hardness at 20° C. ranging from 0.001 to 0.5 MPa, preferably from 0.002 to 0.4 MPa.

The hardness is measured according to a method of penetration of a probe into a sample of compound and in particular using a texture analyser (for example, the TA-XT2i from Rheo) equipped with a stainless steel cylinder with a diameter of 2 mm. The hardness measurement is carried out at 20° C. at the centre of 5 samples. The cylinder is introduced into each sample at a pre-rate of 1 mm/s and then at a measuring rate of 0.1 mm/s, the depth of penetration being 0.3 mm. The value recorded for the hardness is that of the maximum peak.

In addition, this pasty compound is, at a temperature of 23° C., in the form of a liquid fraction and of a solid fraction. In other words, the starting melting temperature of the pasty compound is less than 23° C. The liquid fraction of the pasty compound, measured at 23° C., represents 9 to 97% by weight of the compound. This liquid fraction at 23° C. preferably represents between 15 and 85%, more preferably between 40 and 85%, by weight.

The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the compound to change from the solid state to the liquid state. The pasty compound is “in the solid state” when the whole of its mass is in the crystalline solid form. The pasty compound is “in the liquid state” when the whole of its mass is in the liquid form.

The enthalpy of fusion of the pasty compound is equal to the area under the curve of the thermogram obtained using a differential scanning calorimeter (DSC), such as the calorimeter sold under the name MDSC 2920 by TA Instrument, with a rise in temperature of 5 or 10° C. per minute, according to the ISO Standard 11357-3: 1999. The enthalpy of fusion of the pasty compound is the amount of energy necessary to change the compound from the solid state to the liquid state. It is expressed in J/g.

The enthalpy of fusion consumed at 23° C. is the amount of energy absorbed by the sample to change from the solid state to the state which it exhibits at 23° C., composed of a liquid fraction and of a solid fraction.

The liquid fraction of the pasty compound measured at 32° C. preferably represents from 30 to 100% by weight of the compound, preferably from 80 to 100%, more preferably from 90 to 100%, by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

The pasty compound is preferably chosen from synthetic compounds and compounds of vegetable origin. A pasty compound can be obtained by synthesis from starting materials of vegetable origin.

The pasty compound is advantageously chosen from

polymeric or nonpolymeric silicone compounds

polymeric or nonpolymeric fluorinated compounds

vinyl polymers, in particular:

homopolymers of olefins

copolymers of olefins

hydrogenated homopolymers and copolymers of dienes

homo- or copolymeric, linear or branched, oligomers of alkyl (meth)acrylates preferably having a C₈-C₃₀ alkyl group

homo- and copolymeric oligomers of vinyl esters having C₈-C₃₀ alkyl groups,

homo- and copolymeric oligomers of vinyl ethers having C₈-C₃₀ alkyl groups,

fat-soluble polyethers resulting from the polyetherification between one or more C₂-C₁₀₀, preferably C₂-C₅₀, diols,

esters,

and their mixtures.

The pasty compound is preferably a polymeric compound, in particular a hydrocarbonaceous compound.

Silicone and/or Fluorinated Pasty Compounds

A preferred silicone and fluorinated pasty compound is polymethyl trifluoropropyl methylalkyl dimethylsiloxane, manufactured under the name X22-1088 by Shin Etsu.

When the pasty compound is a silicone and/or fluorinated polymer, the composition advantageously comprises a compatibilizing agent, such as short-chain esters, for example isodecyl neopentanoate.

Polyether Pasty Compounds

Preference is given, among fat-soluble polyethers, in particular to copolymers of ethylene oxide and/or of propylene oxide with long-chain C₆-C₃₀ alkylene oxides, more preferably such that the ratio by weight of the ethylene oxide and/or of the propylene oxide to alkylene oxides in the copolymer is from 5:95 to 70:30. In this family, mention will in particular be made of the copolymers such that the long-chain alkylene oxides are positioned in blocks having an average molecular weight of 1 000 to 10 000, for example a polyoxyethylene/polydodecyl glycol block copolymer, such as the ethers of dodecanediol (22 mol) and of polyethylene glycol (45 EO) sold under the trade name Elfacos ST9 by Akzo Nobel.

Preference is given in particular, among esters, to

esters of vegetable fatty acids, glyceryl isostearate or adipate, sold under the trade name Softisan 649 by Sasol,

arachidyl propionate, sold under the trade name Waxenol 801 by Alzo,

phytosterol esters,

non-crosslinked polyesters resulting from the polycondensation between a linear or branched C₄-C₅₀ dicarboxylic or polycarboxylic acid and a C₂-C₅₀ diol or polyol,

ester aliphatic esters resulting from the esterification of an aliphatic hydroxycarboxylic acid ester by an aliphatic carboxylic acid.

The aliphatic carboxylic acid comprises from 4 to 30 and preferably from 6 to 30 carbon atoms. It is preferably chosen from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, icosanoic acid, isoarachidic acid, octyldodecanoic acid, henicosanoic acid, docosanoic acid, and their mixtures.

The aliphatic carboxylic acid is preferably branched. The aliphatic hydroxycarboxylic acid ester advantageously results from a hydroxylated aliphatic carboxylic acid comprising from 2 to 40 carbon atoms, preferably from 10 to 34 carbon atoms and better still from 12 to 28 carbon atoms, and from 1 to 20 hydroxyl groups, preferably from 1 to 10 hydroxyl groups and better still from 1 to 6 hydroxyl groups. The aliphatic hydroxycarboxylic acid ester is chosen from:

a) partial or complete esters of saturated linear monohydroxylated aliphatic monocarboxylic acids;

b) partial or complete esters of unsaturated monohydroxylated aliphatic monocarboxylic acids;

c) partial or complete esters of saturated monohydroxylated aliphatic polycarboxylic acids;

d) partial or complete esters of saturated polyhydroxylated aliphatic polycarboxylic acids;

e) partial or complete esters of C₂ to C₁₆ aliphatic polyols which have reacted with a mono- or polyhydroxylated aliphatic monocarboxylic or polycarboxylic acid, and their mixtures.

The ester aliphatic esters are advantageously chosen from:

the ester resulting from the esterification reaction of hydrogenated castor oil with isostearic acid in the proportions 1 to 1 (1/1) or hydrogenated castor oil monoisostearate,

the ester resulting from the esterification reaction of hydrogenated castor oil with isostearic acid in the proportions 1 to 2 (½) or hydrogenated castor oil diisostearate,

the ester resulting from the esterification reaction of hydrogenated castor oil with isostearic acid in the proportions 1 to 3 (⅓) or hydrogenated castor oil triisostearate,

and their mixtures.

The choice will preferably be made, among pasty compounds of vegetable origin, of a mixture of soybean sterols and of oxyethylenated (5 EO)/oxypropylenated (5 PO) pentaerythritol sold under the reference Lanolide by Vevy.

The pasty compound preferably represents 1 to 99%, better still 1 to 60%, better still 2 to 30% and even better still 5 to 15% by weight of the composition.

Forms of the Composition

The composition of the invention can be provided in the form of a solid, pasty or liquid composition, the solid composition being compacted or cast as a stick or as a dish. It is advantageously provided in the solid form, namely in the hard form (not flowing under its own weight), in particular in shaped form such as cast or compacted form, for example as a stick or as a dish.

It can be provided in the form of a paste, a solid or cream. It can be an oil-in-water or water-in-oil emulsion, a solid or soft anhydrous gel or in the form of a loose or compacted powder and even in the two-phase form. It is preferably provided in the form of a composition with a continuous oily phase and in particular an anhydrous composition; in this case, it can comprise an aqueous phase at a content of less than 10% and better still of less than 5% by weight.

Colouring Materials

The composition of the invention can advantageously comprise, in addition, at least one colouring material which can be chosen from dyes which are soluble or dispersible in the composition, pigments, pearlescent agents and their mixtures. The dyes are preferably fat-soluble dyes, although water-soluble dyes can be used. This colouring material can represent from 0.001 to 98%, preferably from 0.5 to 85% and better still from 1 to 60% of the total weight of the composition.

For a composition in the form of a paste or in the cast form, such as lipsticks or make-up products for the body, use is generally made of 0.5 to 50% of colouring material, preferably of 2 to 40% and better still of 5 to 30%, with respect to the total weight of the composition.

The fat-soluble dyes are, for example, Soudan Red, D & C Red 17, D & C Green 6, β-carotene, soybean oil, Soudan Brown, D & C Yellow 11, D & C Violet 2, D & C Orange 5, Quinoline Yellow or annnatto. They can represent from 0 to 20% of the weight of the composition and better still from 0.1 to 6%. The water-soluble dyes are in particular beetroot juice and methylene blue and can represent from 0.1 to 6% by weight of the composition (if present).

Preferably, the composition of the invention comprises a particulate phase, advantageously coloured, which can represent from 0.001 to 50% of the total weight of the composition, preferably from 0.01 to 40% and better still from 0.05 to 30%, and which can comprise pigments and/or pearlescent agents and/or fillers commonly used in cosmetic compositions.

The term “pigments” should be understood as meaning white or coloured and inorganic or organic particles which are insoluble in the liquid fatty phase and which are intended to colour and/or opacify the composition. The term “fillers” should be understood as meaning colourless or white, inorganic or synthetic and lamellar or non-lamellar particles. The term “pearlescent agent” should be understood as meaning iridescent particles, in particular produced by certain shell fish in their shells or else synthesized. These fillers and pearlescent agents are used in particular to modify the texture of the composition.

The pigments can be present in the composition in a proportion of 0.05 to 30% (if present) of the weight of the final composition and preferably in the proportion of 2 to 20%. Mention may be made, as inorganic pigments which can be used in the invention, of titanium, zirconium or cerium oxides, zinc, iron or chromium oxides, and ferric blue. Mention may be made, among organic pigments which can be used in the invention, of carbon black and barium, strontium, calcium (D & C Red No. 7) and aluminium lakes.

The pearlescent agents can be present in the composition in a proportion of 0.001 to 20% (if present) of the total weight of the composition, preferably at a level of the order of 1 to 15%. Mention may be made, among pearlescent agents which can be used in the invention, of mica covered with titanium oxide, with iron oxide, with natural pigment or with bismuth oxychloride, such as coloured titanium oxide-coated mica, goniochromatic pigments and, for example, interferential multilayer pigments.

The fillers can be present in a proportion of 0.001 to 35% (if present) of the total weight of the composition, preferably 0.5 to 15%. Mention may in particular be made of talc, mica, kaolin, Nylon® (in particular Orgasol) and polyethylene powders, polytetrafluoroethylene (Teflon®) powders, starch, boron nitride, microspheres formed of copolymers, such as Expancel® (Nobel Industrie), Polytrap® (Dow Corning) or Polypore® L200 (Chemdal Corporation), and silicone resin microbeads (Tospearl® from Toshiba, for example) or silica.

The composition according to the invention can comprise at least one additional nonaqueous compound chosen from oils, fatty substances which are pasty at ambient temperature, waxes, gums, resins, lipophilic polymers and their mixtures.

Waxes and Gums

In particular, the composition can comprise at least one wax.

The term “wax”, within the meaning of the present invention, is understood to mean a lipophilic fatty compound, solid at ambient temperature (25° C.), with a reversible solid/liquid change of state, having a melting point of greater than 30° C. which can range up to 200° C., having a hardness of greater than 0.5 MPa and exhibiting, in the solid state, an anisotropic crystalline arrangement. On bringing the wax to its melting point, it is possible to render it miscible with oils and to form a microscopically homogeneous mixture but, on bringing the temperature of the mixture back to ambient temperature, recrystallization of the wax from the oils of the mixture is obtained.

The waxes which can be used in the invention are compounds which are solid at ambient temperature which are intended to structure the composition, in particular in the stick form; they can be hydrocarbonaceous waxes, fluorinated waxes and/or silicone waxes and can be of vegetable, mineral, animal and/or synthetic origin. In particular, they exhibit a melting point of greater than 40° C. and better still greater than 45° C.

Mention may be made, as wax which can be used in the invention, of those generally used in the cosmetics field: they are in particular of natural origin, such as beeswax, carnauba wax, candelilla wax, ouricury wax, Japan wax, cork fibre wax or sugarcane wax, rice wax, montan wax, paraffin wax, lignite or microcrystalline waxes, ceresin or ozokerite, or hydrogenated oils, such as jojoba oil; synthetic waxes, such as polyethylene waxes resulting from the polymerization or copolymerization of ethylene and Fischer-Tropsch waxes, or esters of fatty acids, such as octacosanyl stearate, glycerides which are solid at 40° C. and better still at 45° C., silicone waxes, such as alkyl or alkoxy dimethicones having an alkyl or alkoxy chain of 10 to 45 carbon atoms, poly(di)methylsiloxane esters which are solid at 40° C., the ester chain of which comprises at least 10 carbon atoms; and their mixtures.

The gums which can be used in the invention are generally provided in the form dissolved in an oil, the polymers are solid at ambient temperature and the resins can be liquid or solid at ambient temperature.

The term “gum” is understood to mean a fatty substance which is provided in the form of a polymer which is solid at ambient temperature having a weight-average molecular weight of 50 000 to 1 000 000. The gum is often sold as a dispersion in an organic solvent of the silicone oil type.

The nature and the amount of the gums or waxes depend on the mechanical properties and on the textures desired. By way of indication, the wax can represent from 0.01 to 50%, preferably from 2 to 40% and better still from 5 to 30% of the total weight of the composition.

The hardness can be measured by the “cheese wire” method, which consists in cutting a stick of lipstick with a diameter of 12.7 mm and in measuring the hardness at 20° C. by means of a DFGHS 2 dynamometer from Indelco-Chatillon moving at a rate of 100 mm/minute. It is expressed as the shear force (expressed in grams) needed to cut a stick under these conditions. According to this method, the hardness of a stick composition according to the invention ranges in particular from 50 to 300 g, preferably from 100 to 250 g and, for example, from 150 to 230 g.

Oils

The term “oil” is understood to mean a fatty substance which is liquid at ambient temperature and atmospheric pressure.

The oils can be hydrocarbonaceous oils and/or silicone oils and/or fluorinated oils. These oils can be of animal, vegetable, mineral or synthetic origin.

The term “hydrocarbonaceous oil” is understood to mean an oil comprising mainly carbon and hydrogen atoms and optionally one or more functional groups chosen from the hydroxyl, ester, ether or carboxyl functional groups. Mention may be made, as example of oils which can be used in the invention, of:

hydrocarbonaceous oils of animal origin, such as perhydrosqualene;

vegetable hydrocarbonaceous oils, such as liquid triglycerides of fatty acids of 4 to 24 carbon atoms, for example triglycerides of heptanoic acid or octanoic acid or sunflower, maize, soybean, cucumber, grape seed, sesame, hazelnut, apricot, macadamia, castor or avocado oils, triglycerides of caprylic/capric acids, such as those sold by Stearineries Dubois or those sold under the names Miglyol 810, 812 and 818 by Dynamit Nobel, jojoba oil or karite butter oil;

linear or branched hydrocarbons of mineral or synthetic origin, such as liquid paraffins and their derivatives, liquid petrolatum, polydecenes or hydrogenated polyisobutene, such as parleam;

synthetic esters and ethers, in particular of fatty acids, such as oils of formula R₁COOR₂ in which R₁ represents the residue of a higher fatty acid comprising from 1 to 40 carbon atoms and R₂ represents a hydrocarbonaceous chain comprising from 1 to 40 carbon atoms with R₁+R₂≧10, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate, or heptanoates, octanoates or decanoates of fatty alcohols; polyol esters, such as propylene glycol dioctanoate, neopentyl glycol diheptanoate or diethylene glycol diisononanoate; and pentaerythritol esters, such as pentaerythrityl tetraisostearate;

fatty alcohols having 12 to 26 carbon atoms, such as octyldodecanol, 2-butyloctanol, 2-hexyldecanol, 2-undecylpentadecanol or oleyl alcohol;

optionally partially hydrocarbonaceous and/or silicone-comprising fluorinated oils;

silicone oils, such as volatile or non-volatile and linear or cyclic polydimethylsiloxanes (PDMS); polydimethylsiloxanes comprising pendent alkyl, alkoxy or phenyl groups or alkyl, alkoxy or phenyl groups at the silicone chain end, groups having from 2 to 24 carbon atoms; or phenylated silicones, such as phenyl trimethicones, phenyl dimethicones, phenyltrimethylsiloxydiphenylsiloxanes, diphenyl dimethicones, diphenylmethyldiphenyltrisiloxanes or (2-phenylethyl)trimethylsiloxysilicates,

their mixtures.

The additional oils can represent from 0 to 90% of the total weight of the composition, preferably from 0.05 to 60% and better still from 1 to 35%.

Preference is given, in the context of the present invention, to additional oils having a molecular weight of between 650 and 10 000 g/mol, preferably of between 750 and 7 500 g/mol.

According to one embodiment, the composition of the invention comprises an oily phase comprising at least 70% by weight of an oil with a molar mass of between 650 and 10 000 g/mol, preferably between 750 and 7 500 g/mol. The oily phase advantageously comprises more than 80%, preferably more than 85%, by weight of an oil with a molar mass of between 650 and 10 000 g/mol, preferably between 750 and 7 500 g/mol.

The oil of high molar mass is preferably chosen from:

lipophilic polymers

esters of linear fatty acids having a total number of carbons ranging from 35 to 70

hydroxylated esters

aromatic esters

branched C₂₄-C₂₈ fatty alcohol or fatty acid esters

silicone oils

oils of vegetable origin

and their mixtures.

The oil of high molar mass is preferably chosen from polybutylenes, hydrogenated polyisobutylenes, polydecenes, hydrogenated polydecenes, vinylpyrrolidone copolymers, such as the PVP/hexadecene copolymer, pentaerythrityl tetrapelargonate, polyglyceryl-2 triisostearate, tridecyl trimellitate, triisoarachidyl citrate, pentaerythrityl tetraisononanoate, pentaerythrityl triisostearate, polyglyceryl-2 tetraisostearate, pentaerythrityl tetra(2-decyltetradecanoate), phenylated silicones, sesame oil, and their mixtures.

Additives

The composition of the invention can additionally comprise any additive conventionally used in the field under consideration, such as water, antioxidants, preservatives, neutralizing agents, lipophilic gelling agents or gelling agents for liquid fatty substances, gelling agents for the aqueous phase, dispersants, or cosmetic or dermatological active principles. These additives, with the exception of water, which can represent from 0 to 70% and, for example, from 1 to 50% and better still from 1 to 10% of the total weight of the composition, can be present in the composition in a proportion of 0 to 20% of the total weight of the composition and better still of 0 to 10%.

The composition according to the invention can be provided in the form of a coloured or colourless composition, in the form of a sun protection composition or make-up removing composition, or in the form of a hygiene composition. It comprises in particular cosmetic active principles. It can then be used as a care or treatment base for the skin, such as the hands or the face, or for the lips (lip balms, protecting the lips from the cold and/or the sun and/or the wind) or a deodorant. Mention may be made, as cosmetic active principle which can be used in the invention, of vitamins A, E, C or B₃, provitamins, such as D-panthenol, soothing active principles, such as α-bisabolol, aloe vera, allantoin, plant extracts or essential oils, protecting or restructuring agents, such as ceramides, freshness active principles, such as menthol and its derivatives, emollients (cocoa butter, dimethicone), moisturizing agents (arginine PCA), anti-wrinkle active principles, essential fatty acids, and their mixtures.

The composition of the invention can also be provided in the form of a product for making up the skin, in particular of the face, such as a foundation, a blusher, a paint, such as a semi-permanent tattooing product or a product for making up the lips, for example a lipstick or a lip gloss, optionally exhibiting care or treatment properties, or a product for making up the superficial body growths, such as, for example, a nail varnish, a mascara, an eyeliner or a product for colouring or caring for the hair.

Of course, the composition of the invention must be cosmetically acceptable, namely be nontoxic and capable of being applied to the skin, superficial body growths or lips of human beings.

The composition according to the invention can be manufactured by known processes generally used in the cosmetic or dermatological field.

A further subject-matter of the invention is the use (i) of at least one ester possessing an aromatic group which is liquid at ambient temperature resulting from the esterification by an aromatic acid of at least one pendent hydroxyl group or hydroxyl group at the chain end of a hydroxylated aliphatic compound chosen from hydroxylated aliphatic acids and their esters and (ii) of at least one pasty compound other than lanolin or of one of its derivatives having a hardness at 25° C. of between 0.001 and 0.5 MPa, preferably of 0.002 to 0.4 MPa, the liquid fraction of which at 23° C. is between 9 and 97% by weight, preferably between 15 and 85%, more preferably between 40 and 85% by weight,

in a composition

for conferring, on a film of the said composition, properties of non-stickiness, of non-greasiness, of gloss, of comfort, of hold over time, of good spreading and/or of slip, and for limiting the exudation of the said composition.

The invention is illustrated in more detail in the following examples. The amounts are given as percentage by mass.

### EXAMPLE 1

### Lipstick

| | |
|---|---|
| Ester of castor oil and of benzoic acid (ratio | 22 |
| 1:1.5) (sold under the reference Finsolv BCO 115 | |
| by Finetex) | |
| Ethers of dodecanediol (22 mol) and of | 11 |
| polyethylene glycol (45 EO) (sold under the | |
| reference Elfacos ST9 by Akzo Nobel) | |
| Triglyceride of 2-decyltetradecanoic acid | 20 |
| Hydrogenated polyisobutene | 10 |
| Diisostearyl malate | 11 |
| Polybutylene | 2.5 |
| Octacosanyl stearate | 5 |
| Mixture of triglycerides | 2 |
| of lauric, myristic, palmitic and stearic acids | |
| (50/20/10/10) | |
| Polyethylene wax (Mw = 500) | 2.5 |
| Polyethylene wax (Mw = 650) | 2.5 |
| Hectorite modified by distearyldimethylammonium | 3 |
| chloride | |
| Pigments | q.s. |
| Preservative | q.s. |
| Fragrance | q.s. |

The oily phase is prepared by mixing the preservative, all the oils (ester of castor oil and of benzoic acid, triglyceride of 2-decyltetradecanoic acid, hydrogenated polyisobutene, diisostearyl malate, polybutylene) and the paste (ethers of dodecanediol (22 mol) and of polyethylene glycol (45 EO)).

Then the hectorite is milled into the oily phase using the triple roll mill (1 passage).

The pigments are subsequently milled into the hectorite+oily phase mixture (3 passages).

The mixture obtained is added to a casserole with the waxes and the mixture is heated at 105° C. for 2 hours while homogenizing using a Raynerie device.

Finally, the fragrance is added, the mixture is homogenized for 5 minutes and then it is cast into a mould at 42° C., which mould is placed at −20° C. for 30 minutes. The sticks are then removed from the mould.

The above formulation has good properties in terms of application (slip), of comfort, of gloss and of hold over time.

### COMPARATIVE EXAMPLE 2

The above formulation was repeated while replacing the pasty compound, composed of 11% of ether of dodecanediol (22 mol) and of polyethylene glycol (45 EO), with 11% of lanolin. The lanolin-free composition according to the invention exhibits greater slip on application and is less sticky while being as glossy. In addition, the film exhibits better hold one hour after the application.

The above written description of the invention provides a manner and process of making and using it such that any person skilled in this art is enabled to make and use the same, this enablement being provided in particular for the subject matter of the appended claims, which make up a part of the original description and including a cosmetic composition for caring for or making up keratinous substances, comprising at least one ester possessing an aromatic group which is liquid at ambient temperature resulting from the esterification by an aromatic acid of at least one pendent hydroxyl group or hydroxyl group at the chain end of a hydroxylated aliphatic compound chosen from hydroxylated aliphatic acids and their esters, the said composition being devoid of lanolin or of lanolin derivatives.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A cosmetic composition for caring for or making up keratinous substances, comprising at least one ester which is liquid at ambient temperature possessing an aromatic group, said ester resulting from the esterification by an aromatic acid of at least one pendent hydroxyl group or hydroxyl group at a chain end of a hydroxylated aliphatic compound selected from the group consisting of hydroxylated aliphatic acids and their esters, and mixtures thereof, said composition being devoid of lanolin and lanolin derivatives.

2. The composition according to claim 1, further comprising at least one pasty compound other than lanolin or than one of its derivatives.

3. The composition according to claim 1, wherein the aromatic acid of the ester possessing an aromatic group is selected from the group consisting of benzoic acid, phenylacetic acid, cinnamic acid, 3-phenylpropanoic acid, salicylic acid, terephthalic acid, trimellitic acid, pyromellitic acid and mixtures thereof.

4. The composition according to claim 1, wherein the aromatic acid is benzoic acid.

5. The composition according to claim 1, wherein the hydroxylated aliphatic acids comprise from 2 to 40 carbon atoms.

6. The composition according to claim 1, wherein the hydroxylated aliphatic acids additionally comprise from 1 to 20 hydroxyl groups capable of being esterified by the aromatic acid.

7. The composition according to claim 1, wherein the hydroxylated aliphatic compound is selected from the group consisting of:
i) saturated linear monohydroxylated aliphatic monoacids of formula: 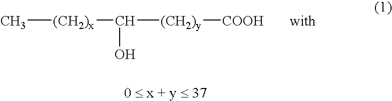
or (2) HO—CH₂—(CH₂)ₓ—COOH with 0<x<38;
ii) saturated branched monohydroxylated aliphatic monoacids of formula: 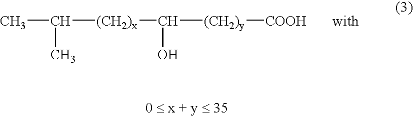
or (3′) 2-ethyl-3-hydroxycaprylic acid of formula: 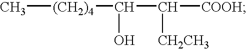
iii) unsaturated monohydroxylated aliphatic monoacids of formula: 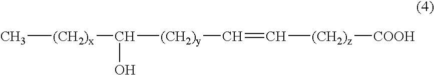
with 0≦x+y+z≦35 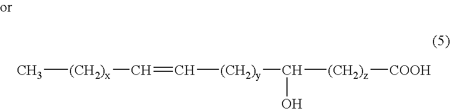
with 0≦x+y+z≦35
or (6) HOCH₂—(CH₂)ₙ—CH═CH—(CH₂)_{y}—COOH with 0≦x+y≦36;
iv) saturated polyhydroxylated aliphatic monoacids of formula: 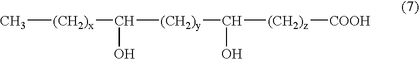
with 0≦x+y+z≦36;
v) saturated monohydroxylated aliphatic polyacids of formula: 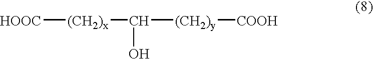
with 0≦x+y≦37;
vi) saturated polyhydroxylated aliphatic polyacids;
vii) esters of saturated linear monohydroxylated aliphatic monoacids;
viii) esters of unsaturated monohydroxylated aliphatic monoacids;
x) esters of saturated polyhydroxylated aliphatic polyacids;
xi) partial or complete esters of C₂ to C₁₆ polyol which has reacted with a hydroxylated aliphatic acid;
and mixtures thereof.

8. The composition according to claim 1, wherein the hydroxylated aliphatic compound is selected from the group consisting of:
lactic acid; 12-hydroxyoctadecanoic acid; α-hydroxyoctadecanoic acid;
glycolic acid or juniperic acid;
leucinic acid or 2-ethyl-3-hydroxycaprylic acid;
ricinoleic acid;
-3-hydroxy-4-hexenoic acid or hydroxynervonic acid;
-16-hydroxy-6-hexadecenoic acid;
-9,10-dihydroxyoctadecanoic acid, 9,12-dihydroxyoctadecanoic acid, aleuritic acid, 9,10,12-trihydroxyoctadecanoic acid, hexahydroxyoctadecanoic acid or octahydroxyoctadecanoic acid;
malic acid or citric acid;
tartaric acid;
isostearyl lactate, the lactate resulting from C₁₂-C₁₃ alcohol, octyldodecyl lactate, oleyl lactate, myristyl lactate;
2-ethylhexyl hydroxystearate, octyldodecyl hydroxystearate, isostearyl hydroxystearate, isodecyl hydroxystearate, glyceryl trihydroxystearate, dipentaerythrityl hexahydroxystearate;
butyl ricinoleate, octyldodecyl ricinoleate, cetyl ricinoleate, glyceryl triricinoleate;
diisostearyl malate, triisostearyl citrate, trioctyldodecyl citrate;
the tartrate resulting from branched C₁₂-C₁₃ dialcohols;
and mixtures thereof.

9. The composition according to claim 1, wherein the aromatic ester is an aliphatic fatty acid ester ester, the fatty acid residue of which comprises at least 12 carbon atoms.

10. The composition according to claim 1, wherein the hydroxylated aliphatic compound is selected from the group consisting of esters of ricinoleic acid, esters of 12-hydroxystearic acid, esters of lactic acid, esters of 14-hydroxyicosenoic acid, and mixtures thereof.

11. The composition according to claim 1, wherein the aromatic ester exhibits a viscosity of greater than 500 cP (50 Pa·s) at 20° C. and/or a refractive index ≧1.48.

12. The composition according to claim 1, wherein the aromatic ester is selected from the group consisting of glyceryl monobenzoyl ricinoleate, glyceryl mono/dibenzoyl ricinoleate, glyceryl dibenzoyl ricinoleate, glyceryl tribenzoyl ricinoleate, and mixtures thereof.

13. The composition according to claim 1, wherein the aromatic ester is present in an amount sufficient to confer, on the composition, properties of non-greasiness, of non-stickiness, of slip, of gloss, of coverage, of non-exudation and/or of hold over time.

14. The composition according to claim 1, wherein the aromatic ester is present in an amount ranging from 5 to 90% of the total weight of the composition.

15. The composition according to claim 2, wherein the pasty compound has a hardness at 20° C. of between 0.001 and 0.5 MPa

16. The composition according to claim 2, wherein the pasty compound has a liquid fraction at 23° C. of between 40 and 85%, by weight.

17. The composition according to claim 2, wherein the pasty compound has a liquid fraction at 32° C. of between 90 and 100%, by weight.

18. The composition according to claim 2, wherein the pasty compound is selected from the group consisting of
polymeric or nonpolymeric silicone compounds
polymeric or nonpolymeric fluorinated compounds
vinyl polymers, as follows:
homopolymers of olefins
copolymers of olefins
hydrogenated homopolymers and copolymers of dienes
homo- or copolymeric, linear or branched, oligomers of alkyl (meth)acrylates preferably having a C₈-C₃₀ alkyl group
homo- and copolymeric oligomers of vinyl esters having C₈-C₃₀ alkyl groups,
homo- and copolymeric oligomers of vinyl ethers having C₈-C₃₀ alkyl groups,
fat-soluble polyethers resulting from the polyetherification between one or more C₂-C₁₀₀, preferably C₂-C₅₀, diols,
esters,
and mixtures thereof.

19. The composition according to claim 2, wherein the pasty compound is a hydrocarbonaceous compound.

20. The composition according to claim 18, wherein the pasty compound is polymethyl trifluoropropyl methylalkyl dimethylsiloxane.

21. The composition according to claim 18, wherein the fat-soluble polyether is selected from the group consisting of copolymers of ethylene oxide and/or of propylene oxide with long-chain C₆-C₃₀ alkylene oxides.

22. The composition according to claim 21, wherein the fat-soluble polyether is a polyoxyethylene/polydodecyl glycol block copolymer.

23. The composition according to claim 18, wherein the esters are selected from the group consisting of
esters of vegetable fatty acids,
arachidyl propionate,
phytosterol esters,
non-crosslinked polyesters resulting from the polycondensation between a linear or branched C₄-C₅₀ dicarboxylic or polycarboxylic acid and a C₂-C₅₀ diol or polyol,
ester aliphatic esters resulting from the esterification of an aliphatic hydroxycarboxylic acid ester by an aliphatic carboxylic acid,
and mixtures thereof.

24. The composition according to claim 23, wherein the aliphatic carboxylic acid of the aliphatic ester is selected from the group consisting of hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, icosanoic acid, isoarachidic acid, octyldodecanoic acid, henicosanoic acid, docosanoic acid, and mixtures thereof.

25. The composition according to claim 23, wherein the aliphatic hydroxycarboxylic acid ester results from a hydroxylated aliphatic carboxylic acid comprising from 2 to 40 carbon atoms and from 1 to 20 hydroxyl groups.

26. The composition according to claim 25, wherein the aliphatic hydroxycarboxylic acid ester is selected from the group consisting of:
a) partial or complete esters of saturated linear monohydroxylated aliphatic monocarboxylic acids;
b) partial or complete esters of unsaturated monohydroxylated aliphatic monocarboxylic acids;
c) partial or complete esters of saturated monohydroxylated aliphatic polycarboxylic acids;
d) partial or complete esters of saturated polyhydroxylated aliphatic polycarboxylic acids;
e) partial or complete esters of C₂ to C₁₆ aliphatic polyols which have reacted with a mono- or polyhydroxylated aliphatic monocarboxylic or polycarboxylic acid,
and mixtures thereof.

27. The composition according to claim 26, wherein the aliphatic hydroxycarboxylic acid ester is selected from the group consisting of:
the ester resulting from the esterification reaction of hydrogenated castor oil with isostearic acid in the proportions 1 to 1 (1/1) or hydrogenated castor oil monoisostearate,
the ester resulting from the esterification reaction of hydrogenated castor oil with isostearic acid in the proportions 1 to 2 (½) or hydrogenated castor oil diisostearate,
the ester resulting from the esterification reaction of hydrogenated castor oil with isostearic acid in the proportions 1 to 3 (⅓) or hydrogenated castor oil triisostearate,
and mixtures thereof.

28. The composition according to claim 1, wherein it furthere comprises an oily phase comprising at least 70% by weight of an oil with a molar mass of between 650 and 10 000 g/mol.

29. The composition according to claim 28, wherein the oil of high molar mass is selected from the group consisting of:
lipophilic polymers
esters of linear fatty acids having a total number of carbons ranging from 35 to 70
hydroxylated esters
aromatic esters
branched C₂₄-C₂₈ fatty alcohol or fatty acid esters
silicone oils
oils of vegetable origin
and mixtures thereof.

30. The composition according to claim 28, wherein the oil of high molar mass is selected from the group consisting of polybutylenes, hydrogenated polyisobutylenes, polydecenes, hydrogenated polydecenes, vinylpyrrolidone copolymers, pentaerythrityl tetrapelargonate, polyglyceryl-2 triisostearate, tridecyl trimellitate, triisoarachidyl citrate, pentaerythrityl tetraisononanoate, pentaerythrityl triisostearate, polyglyceryl-2 tetraisostearate, pentaerythrityl tetra(2-decyltetradecanoate), phenylated silicones, sesame oil, and mixtures thereof.

31. The composition according to claim 28, wherein it comprises an oily phase comprising at least 80% of an oil with a molar mass of between 650 and 10 000 g/mol.

32. The composition according to claim 1, which is provided in the form of a product for making up the body, of a lipstick, of a lip gloss, of a mascara, of a nail varnish, of a product for colouring or caring for the hair, or of a deodorant.

33. The composition according to claim 1, wherein it further comprises at least one additional fatty substance chosen from oils, waxes, gums, resins, lipophilic polymers and their mixtures.

34. The composition according to claim 1, wherein it further comprises at least one colouring material.

35. The composition according to claim 34, wherein the colouring material is selected from the group consisting of dyes which are soluble or dispersible in the composition, pigments, pearlescent agents and their mixtures.

36. The composition according to claim 1, wherein it further comprises an additive selected from the group consistinf of antioxidants, cosmetic or dermatological active principles, preservatives, gelling agents for liquid fatty substances, dispersants and mixtures thereof.

37. The composition according to claim 1, wherein it further comprises a cosmetic active principle selected from the group consisting of vitamins A, E, C or B₃, provitamins, soothing active principles, aloe vera, allantoin, plant extracts or essential oils, protecting or restructuring agents, freshness active principles, emollients, moisturizing agents, anti-wrinkle active principles, essential fatty acids, and mixtures thereof.

38. The composition according to claim 1, wherein it is provided in shaped form.

39. The composition according to claim 1, wherein it is provided in the form of a continuous oily phase.

40. The composition according to claim 1, wherein it is provided in the form of a lipstick.

41. A method for conferring to a composition properties of gloss, of comfort, of hold over time, of non-stickiness, of non-greasiness, of good spreading and/or of slip, and/or for limiting the exudation of the said composition, comprising combining therewith (i) of at least one ester possessing an aromatic group which is liquid at ambient temperature resulting from the esterification by an aromatic acid of at least one pendent hydroxyl group or hydroxyl group at the chain end of a hydroxylated aliphatic compound chosen from hydroxylated aliphatic acids and their esters and (ii) of at least one pasty compound other than lanolin or of one of its derivatives having a hardness at 25° C. of between 0.001 and 0.5 MPa, the liquid fraction of which at 23° C. is between 9 and 97% by weight.
